(19) United States
(12) Patent Application Publication
(10) Pub. No.: US 2007/0021837 A1
(43) Pub. Date: Jan. 25, 2007

(54) Stabilizing augment for prosthetic disc

(76) Inventor: Richard B. Ashman, New Orleans, LA (US)

Correspondence Address: MAGINOT, MOORE & BECK, LLP;CHASE TOWER, 111 MONUMENT CIRCLE, SUITE 3250, INDIANAPOLIS, IN, 46204, US

(21) Appl. No.: 11/489,765

(22) Filed: Jul. 19, 2006

Related U.S. Application Data
(60) Provisional application No. 60/700,978, filed on Jul. 20, 2005.

(51) Int. Cl. A61F 2/44

(52) U.S. Cl. 623/17.16; 623/17.15

(57) ABSTRACT

An adjunct device for use with a disc replacement prosthesis engages the prosthesis to restrict or prevent relative movement or articulation between the end plates of the disc prosthesis. The device is configured to be implanted within an existing disc prosthesis in lieu of removal and replacement of the prosthesis or fusion of the motion segment.

### BACKGROUND OF THE INVENTION

The present invention relates generally to the field of spinal implant systems, and particularly to artificial disc replacement systems. More particularly, the invention concerns enhancements to modify or adjust the extent or degrees of freedom of an articulating arthroplasty implant.

Arthrodesis is the surgical immobilization of a joint, often termed joint fusion. On the other hand, arthroplasty is the surgical repair of a joint so that the joint retains as much of its original mobility as possible. Arthroplasty, which is quickly replacing arthrodesis as the standard treatment of spinal disc problems, is achieved using two general types of arthroplasty implants. A nucleus implant replaces only the nucleus pulposus, while a prosthetic disc replaces the entire disc including the annulus. Several articulating prosthetic discs have been developed for use in replacing diseased, injured, or degenerating discs in patients.

One common artificial disc design is depicted in FIGS. 1 and 2. This artificial disc replacement 10 a superior end plate 11 and an inferior end plate 12 that contact the endplates of the adjacent vertebral bodies when placed in the space previously occupied by the disc. The end plates 11, 12 may have roughened surfaces 13, 14 or may incorporate teeth or protrusions 15 for securing the end plates to the vertebral body. With this type of prosthesis, each end plate has a corresponding convex bearing interior surface 16, 17 with an intermediate component 18 sandwiched between and bearing on the surfaces. In one version of the prosthesis, the intermediate component is an articulating bearing element. In another versions, the intermediate component may be a cushion or elastically deformable element. Different methods of articulation and modes of fit for the articulating component may be employed in the myriad artificial disc prostheses.

In accordance with one type of prosthesis, the intermediate component 18 is an articulating component configured as shown in FIG. 2 with superior and inferior convex surfaces 19, 20 that contact the corresponding concave bearing surfaces 16, 17 of the superior and inferior end plates. The articulating component 18 of some disc prostheses uses a cushion, or an elastomeric or compressible material, while others use generally rigid material.

In some cases, arthrodesis may be indicated, even after the implantation of an arthroplasty artificial disc. For instance, arthrodesis may be recommended due to significant changes in bone or muscle over time, implant wear, or recurrence of troublesome symptoms. Because many of the artificial disc designs promote bone growth into the endplates of the prosthesis for stability and longevity, removal of the artificial endplates may be difficult and may require significant surgical invasion and loss of bone mass from the adjacent vertebrae. Furthermore, most spinal fusions are achieved using implants that may need to be positioned in the space taken up by the disc replacement.

What is needed is a way to stabilize the artificial disc or to modify its motion characteristics without the need to remove the artificial endplates or the disc prosthesis.

### SUMMARY OF THE INVENTION

To address this need, the present invention contemplates devices and enhancements that will allow the superior and inferior end plates of the original disc replacement prosthesis to remain implanted where a reduction in range of articulation or even arthrodesis is indicated. The invention contemplates fixing or limiting the range of motion of the disc prosthesis in a manner similar to that of arthrodesis through alterations to the artificial disc prosthesis itself. The features of present invention may be used with a wide range of disc prosthesis designs, including designs incorporating articulating bearing and designs utilizing a cushion or elastomeric element.

The invention contemplates adjunct components that may be used singly or in conjunction with one another to restrict articulation of or demobilize the disc prosthesis. The motion of the adjacent vertebrae can be fixed or limited by varying the geometry of the adjunct components. Furthermore, the superior-inferior separation and angle (lordotic or kyphotic) can be fixed using the adjunct components.

One embodiment of the invention comprises a replacement for the articulating component of the implant which seats within the bearing surfaces of the superior and inferior end plates of the prosthesis in the same manner as the original articulating component. The replacement component is configured to limit the relative movement between the end plates of the prosthesis. In one aspect, the replacement component may include an upstanding rim that contacts the interior face of an end plate so that no articulation can occur. A variation of this embodiment is to limit the amount of articulation that can occur in any given direction by changing the geometry of the replacement component to allow some motion before the face of either end plate contacts the rim.

A further embodiment of the invention includes a component that can be inserted around the articulating component of the prosthesis which fixes the angle and/or separation of the disc prosthesis end plates and/or limit the relative movement or articulation of the artificial disc. This adjunct component may be a collar of a predetermined geometry to fix or limit the articulation of the disc as well as fix the separation and angle of the space.

Limitation of the angle, separation, and/or articulation may further be achieved through the use of adjunct hardware attached to the disc prosthesis. In one embodiment, the end plates are configured to receive fasteners that are used to engage a stabilizing element, such as a plate or strut. In a variation of this embodiment, a replacement for the articulating component can incorporate the mounting features.

One object of the invention is to fix or limit the movement of the adjacent vertebrae with respect to each other without removing the end plates of the artificial disc. Yet another object of the invention is to fix the movement of the vertebrae without the need for bone grafting or alternatively, to hold the vertebrae in place to allow a bone graft to heal.

One benefit of the present invention is that it may be implanted within an existing disc prosthesis previously implanted in the patient. Another benefit is that the devices of the invention may be tailored to prevent all relative movement or permit limited relative movement between the prosthesis end plates. Other objects and benefits of the invention will become apparent upon consideration of the following written description taken together with the accompanying figures.

### DESCRIPTION OF THE FIGURES

FIG. 1 is a front perspective view of a typical artificial total disc replacement prosthesis.

FIG. 2 is a front perspective view of one typical semi-spherical intermediate component of the total disc replacement prosthesis shown in FIG. 1.

FIG. 3 is a front perspective view of a replacement for the articulating element, according to one embodiment of the invention, configured for use in total disc replacement prosthesis shown in FIG. 1.

FIG. 4 is a front perspective view of an implant according to a further embodiment of the invention which is placed around the articulating element of the artificial total disc replacement prosthesis shown in FIG. 1 to fix the angle and maintain separation of the end plate components of the prosthesis.

FIG. 5 is a top elevational view of the implant shown in FIG. 4.

FIG. 6 is a front elevational view of the implant shown in FIG. 4.

FIG. 7 is a side elevational view of the implant shown in FIG. 4.

FIG. 8 is a front perspective view of an implant according to an additional embodiment of the invention that mates with an artificial total disc replacement prosthesis modified from the prosthesis shown in FIG. 1.

FIG. 9 is a front perspective view of a variation of implant shown in FIG. 8 according to a further embodiment of the invention.

FIG. 10 is a front perspective view of an implant according to another embodiment of the invention that is adapted to mate with the intermediate component of a total disc replacement prosthesis modified from the prosthesis shown in FIG. 1.

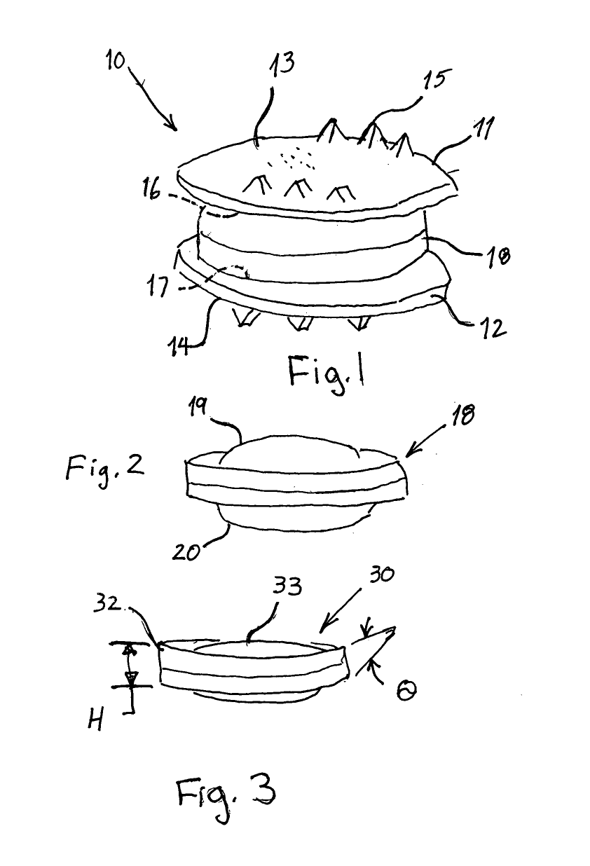

### DESCRIPTION OF THE PREFERRED EMBODIMENTS

For the purposes of promoting an understanding of the principles of the invention, reference will now be made to the embodiments illustrated in the drawings and described in the following written specification. It is understood that no limitation to the scope of the invention is thereby intended. It is further understood that the present invention includes any alterations and modifications to the illustrated embodiments and includes further applications of the principles of the invention as would normally occur to one skilled in the art to which this invention pertains.

The present invention provides devices to restrict or modify the articulation or range of motion of an implanted disc prosthesis. Thus, the devices of the invention may be used in lieu of removal of the disc prosthesis and either replacing the prosthesis or fusing the motion segment.

In accordance with one embodiment of the invention, a replacement component 30, shown in FIG. 3, is configured to replace the intermediate component 18 of a disc prosthesis, such as the prosthesis 10 depicted in FIG. 1. The replacement component is similar in construction to the component it is replacing, but incorporates an extended rim 32 that projects away from the superior bearing surface 33 that is configured to mate with the bearing surface 16 of the superior end plate 11. The rim 32 is configured to contact the underside of the superior end plate 11 to inhibit articulation. The height H or angle θ of the rim 32 can be modified to allow for a limited degree of articulation if appropriate. The height H and angle θ of the rim may also be modified at different locations around the rim to produce the appropriate level of separation and curvature for the location of the artificial disc. For instance, the height and/or angle may be greater at the anterior side of the component 30 than at the posterior side.

In the illustrated embodiment, the rim 32 extends toward the superior end plate 11, thereby shrouding all but a portion of the superior bearing surface 33 that contacts the bearing surface 16 of the superior end plate. The rim may also extend toward the inferior end plate 12 in the same manner. Preferably, the rim 32 is sized to retain some portion of the bearing surfaces of the replacement intermediate component 30 so that the bearing surfaces can help position the component between the two end plates of the prosthesis.

It can be appreciated that by replacing the articulating component of the artificial disc with an appropriately dimensioned component 30 articulation of the modified disc prosthesis 10 may be limited or fixed without removing or replacing the superior and inferior end plates 11, 12 of the artificial disc replacement. The subject disc space may be distracted to allow removal of the original intermediate component 18 and insertion of the replacement component 30. When the distraction is released, the replacement component seats within the bearing surfaces 16, 17 of the two plates.

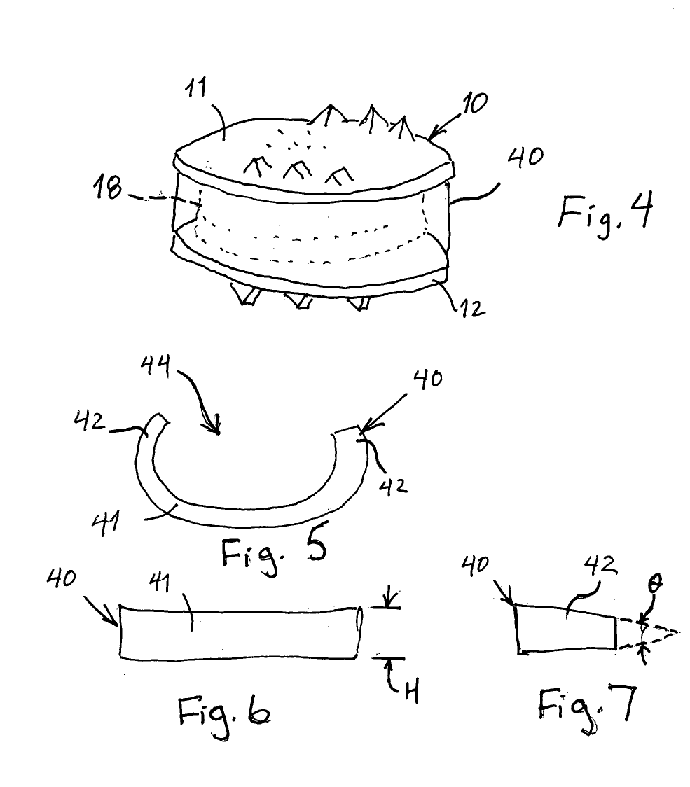

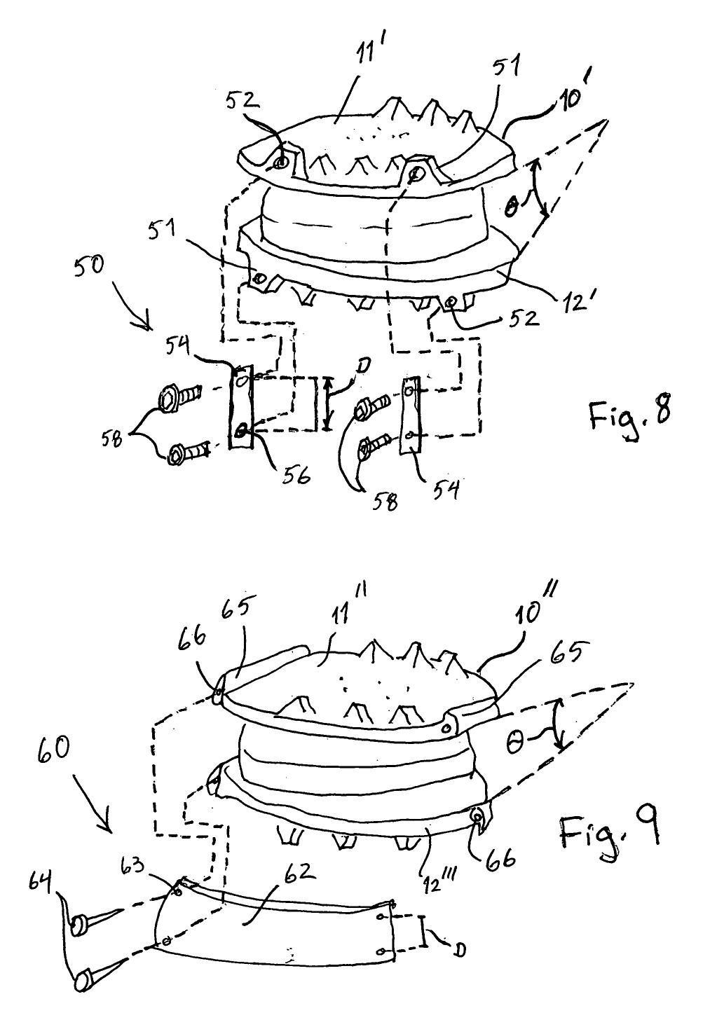

FIGS. 4-7 show another embodiment of the invention incorporating a collar 40 that may be inserted around the intermediate component 18 of the artificial disc prosthesis 10. The collar 40 is made to a predetermined height H at its greatest dimension that is calibrated to prevent or limit articulation of the superior end plate 11 relative to the inferior end plate 12. The body 41 of the collar is configured with a generally C-shaped opening 44 to partially encircle the intermediate component 18, preferably greater than 180° around the component so that the collar may be physically fixed in position around the component. In a specific embodiment, the collar spans about 210°. The body 41 includes side wings 42 that may be flexed slightly outward as the collar is placed around the intermediate component 18. It is contemplated that at least the wings 42 are formed of a resilient material that may be flexed as the body 41 is inserted within the disc prosthesis, but return to their original C-shaped configuration to fit snugly about the intermediate component 18. As shown in FIG. 7, the side wings 42 may be formed to an appropriate angle θ to account for lordosis, of example, at the particular vertebral level.

The collar 40 may be configured to restrict or prohibit articulation of the disc prosthesis 10. If the height H of the collar is substantially equal to the distance between the superior and inferior end plates 11, 12, the collar will prevent articulation. If the height is less, some articulation may be permitted until the two plates contact the collar.

In an alternative embodiment of the invention, the superior and inferior end plates are coupled by an assembly 50 to restrict or prevent their movement relative to each other. Thus, in one embodiment shown in FIG. 8 the plates 11′ and 12′ of a modified disc prosthesis 10′ are provided with a mounting boss 51 defining fastener holes 52. The mounting bosses 51 may be integrated into the side of the plates 11′, 12′ or may be formed as tabs extending away from the plates. In this latter case, the tabs may contact the outer surface of the vertebral body to which the plates are affixed.

In accordance with a further feature of this embodiment, stabilizing elements 54 are provided that are sized to span between pairs of fastener holes 52 of the two plates. The stabilizing elements may be struts or elongated plates that are provided with openings 56 for receiving an appropriate fastener 58, which may preferably be in the form of set screws that extend through the openings 56 and into the fastener holes 52. In one specific embodiment, the openings 56 are sized to exactly receive the fasteners 58 so that the struts 54 prevent any relative movement between the superior and inferior end plates 11′, 12′. In another specific embodiment, the openings 56 may be slots elongated along the length of the struts so that some relative movement is permitted as the fasteners 58 ride within the elongated openings.

It can be appreciated that the struts could be replaced by other stabilizing elements and the mounting method could be any other suitable fastener other than screws. One such variation is shown in FIG. 9, in which an assembly 60 includes a stabilizing element in the form of an elongated plate 62 with openings 63 arranged to mate with corresponding fastener holes 66 defined in the superior and inferior end plates 11″, 12″ of a modified disc prosthesis 10″. The fastener holes 66 may be defined in a boss 65 that extends from the respective plate. Fasteners 64 extend through the openings 63 into the fastener holes 66 to fix the plate 62 to the disc prosthesis 10″. The fasteners may be set screws or pins, as illustrated in FIG. 9. The pins are preferably configured for press-fit engagement within the corresponding fastener hole 66. The stabilizing plate 62 is curved as necessary to conform to the profile of the end plates 11′, 12′ of the disc prosthesis and fit within the space between the end plates.

It can also be appreciated that in both of these embodiments, the distance D between the mounting openings 63 on the plate 62 establish the separation between the superior and inferior end plates 11″, 12″ of the disc prosthesis 10″. This distance may produce an angle θ between the superior and inferior end plates to account for spinal curvature at the particular vertebral level.

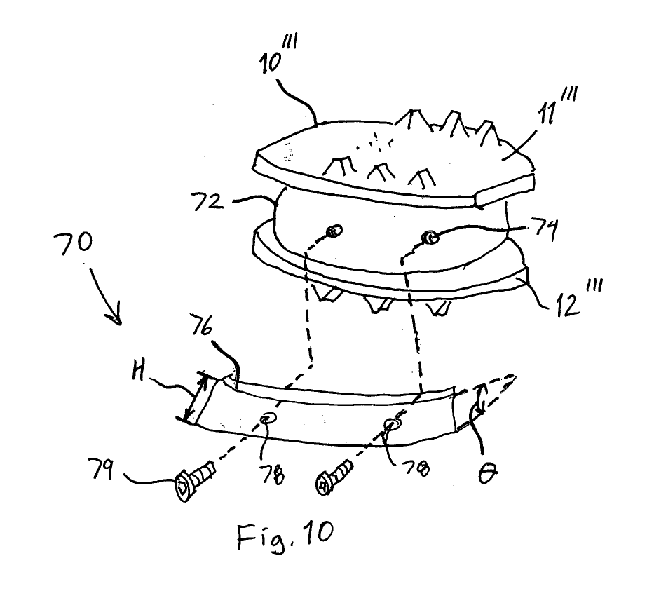

FIG. 10 depicts yet another embodiment of the invention. This embodiment contemplates an assembly 70 utilizes a modified intermediate component 72 as part of the disc prosthesis 10″′. In this embodiment, the intermediate component 72 incorporates fastener holes 74 that can be used to attach a structural member 76 thereto. The structural member, or plate 76 defines mounting openings 78 configured to receive fasteners 79 for engagement with the holes 74. Preferably, the fasteners 79 are set screws, but other suitable fasteners may be provided for fastening the plate 76 to the intermediate component 72. It can be appreciated that the height H of the plate 76 may be calibrated to prevent or restrict relative motion between the superior and inferior end plates 11″′, 12″′. The plate may also be angled at an angle θ to introduce a fixed angle between the two plates of the disc prosthesis 10″′. As with the previous embodiment, the dimensions of the plate 76 may be adjusted so that the superior and inferior end plates bottom against the plate 76 after a limited range of motion.

It is contemplated that the adjunct components of the present invention are formed of biocompatible materials with sufficient strength to effectively prevent or restrict movement of the previously implanted disc prosthesis. Thus, the replacement intermediate component 30 may be formed of the same material as the original intermediate component 18 forming part of the originally implanted disc prosthesis. The collar 40 shown in FIGS. 4-7 may be formed of a similar material as the intermediate component, or may be formed of a high-density polymer or a medical grade metal. The struts 54, plate 62 and plate 76 may be formed of the same material. It is understood that the material preferably does not buckle between the superior and inferior end plates of the disc prosthesis under expected spinal loads. However, in some cases, the material may buckle but resilient return to its original shape once the spinal load is removed.

While the invention has been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

For example, while the stabilizing elements 54, 62, and 76 are engaged to the corresponding component of the disc prosthesis, other forms of engagement are contemplated. In an alternative embodiment, mounting pins may extend directly from the stabilizing elements instead of separate fasteners. The mounting pins may be configured to form a press-fit engagement with the respective fastener holes. Alternatively, the pins may project from the respective component of the disc prosthesis, although this approach is less desirable since surrounding tissue may encumber access to the pins for engagement with the stabilizing element(s). It should be understood, however, that the manner of engagement or fixation of the stabilizing elements to the end plates or intermediate component of the disc prosthesis take on any form contemplated by a person of ordinary skill in the art of spinal fixation or disc prostheses.

In a further modification of the embodiments utilizing the stabilizing elements shown in FIG. 8 or 9, the stabilizing elements may be modified to engage only one of the end plates. In this modification, the stabilizing elements 54 or 62 may be configured for contact with, but not fixation to, the other end plate.

What is claimed is:

1. A device for use with a disc prosthesis implanted within a disc space, the prosthesis having an intermediate element disposed between a superior end plate and an inferior endplate affixed to the opposing superior and inferior vertebrae, respectively, said device operable to limit the range of motion of the end plates relative to each other, said device comprising:
a replacement intermediate component configured to fit between the superior and inferior end plates of the disc prosthesis in situ within the disc space,
said intermediate component defining a rim disposed between the superior and inferior end plates of the prosthesis and having a height sized so that said rim is contacted by at least one of the superior and inferior end plates of the prosthesis as the end plates move relative to each other.

2. The device of claim 1, wherein said height of said rim is sized to substantially span the height of the disc space between the end plates of the prosthesis, to thereby prevent relative movement between the end plates.

3. The device of claim 1, wherein said height of said rim is sized to span only a portion of the height of the disc space between the end plates, to thereby permit restricted relative movement between the end plates that is less than the relative movement in the absence of said rim.

4. The device of claim 1, wherein said rim is angled to establish a predetermined angle between the superior and inferior end plates when the end plates contact said rim.

5. A device for use with a disc prosthesis implanted within a disc space, the prosthesis having an intermediate element disposed between a superior end plate and an inferior endplate affixed to the opposing superior and inferior vertebrae, respectively, said device operable to limit the range of motion of the end plates relative to each other, said device comprising:
a collar configured to be positioned between the end plates of the disc prosthesis in situ within the disc space, said collar having a substantially C-shaped body sized to partially encircle the intermediate element of the prosthesis when the collar is positioned between the end plates.

6. The device of claim 5, wherein at least a portion of said collar is resiliently deformable to fit around the intermediate component of the disc prosthesis when the collar is positioned between the end plates.

7. The device of claim 5, wherein at least a portion of said collar is angled to establish a predetermined angle between the superior and inferior end plates when the end plates contact said collar.

8. The device of claim 5, wherein said collar has a height that is sized to substantially span the height of the disc space between the end plates of the prosthesis, to thereby prevent relative movement between the end plates.

9. The device of claim 5, wherein said collar has a height that is sized to span only a portion of the height of the disc space between the end plates, to thereby permit restricted relative movement between the end plates that is less than the relative movement in the absence of said collar.

10. A device for use with a disc prosthesis implanted within a disc space, the prosthesis having an intermediate element disposed between a superior end plate and an inferior endplate affixed to the opposing superior and inferior vertebrae, respectively, said device operable to limit the range of motion of the end plates relative to each other, said device comprising:
at least one stabilizing element sized to span between the superior and inferior end plates of the disc prosthesis; and
a fastener assembly for engaging each of said at least one stabilizing element to at least one of the end plates or intermediate component of the disc prosthesis.

11. The device of claim 10, wherein:
the at least one stabilizing element includes at least two elongated struts sized to span between the superior and inferior end plates; and
said fastener assembly includes;
a fastener hole defined in at least one of the end plates for each of said at least two struts;
an opening defined in one end of each of said struts; and
a fastener configured to extend through said opening in each of said struts for engagement within a corresponding fastener hole in the end plate.

12. The device of claim 11, wherein said fastener assembly further includes;
a fastener hole defined in both of the end plates for each of said at least two struts;
an opening defined in each end of each of said struts; and
a fastener configured to extend through each opening in each of said struts for engagement within a corresponding fastener hole in each end plate.

13. The device of claim 10, wherein:
the at least one stabilizing element is an elongated plate sized to span between the superior and inferior end plates; and
said fastener assembly includes;
at least a pair of fastener holes defined in at least one of the end plates;
at least one opening defined at each end of said plate; and
at least a pair of fasteners configured to extend through a corresponding one of said at least one opening in each end of said plate for engagement within a corresponding fastener hole in the end plate.

14. The device of claim 13, wherein said fastener assembly further includes;
at least a pair of fastener holes defined in both of the end plates;
at least a pair of openings defined at each end of said plate; and
a fastener configured to extend through each opening for engagement within a corresponding fastener hole in each end plate.

15. The device of claim 10, wherein said stabilizing element is angled to establish a predetermined angle between the superior and inferior end plates when said stabilizing element is engaged to the disc prosthesis.

16. The device of claim 10, wherein:
the at least one stabilizing element is an elongated plate sized to span between the superior and inferior end plates; and
said fastener assembly includes;
at least one fastener hole defined in the intermediate element of the disc prosthesis;
at least one opening defined in said plate; and
at least one fastener configured to extend through said at least one opening for engagement within a corresponding fastener hole in the intermediate element.
